(19) United States
(12) Patent Application Publication
(10) Pub. No.: US 2020/0038680 A1
(43) Pub. Date: Feb. 6, 2020

(54) Optical Stimulation Arrangement

(71) Applicant: University of Newcastle Upon Tyne, Tyne and Wear (GB)

(72) Inventors: Patrick Degenaar, Tyne and Wear (GB); Fahimeh Dehkhoda, Tyne and Wear (GB); Ahmed Soltan, Tyne and Wear (GB); Hubin Zhao, Tyne and Wear (GB); Reza Ramezani, Tyne and Wear (GB)

(73) Assignee: University of Newcastle Upon Tyne, Tyne and Wear (GB)

(21) Appl. No.: 16/338,423

(22) PCT Filed: Sep. 29, 2017
(86) PCT No.: PCT/EP2017/074866

§ 371 Date: Mar. 29, 2019

(30) Foreign Application Priority Data
Sep. 30, 2016 (GB) 1616725.6

(51) Int. Cl. A61N 5/06

(52) U.S. Cl. CPC A61N 5/0622; A61N 2005/067; A61N 2005/0651; A61N 5/0601

(57) ABSTRACT

An optical stimulation arrangement including a light-emitting device, implantable in an environment with an associated ground voltage, the light emitting device including: a light emitting element; an anode; and a cathode; and a controller for driving the light-emitting device in a biphasic manner.

### FIELD OF THE INVENTION

The present invention relates to an optical stimulation arrangement including a light-emitting element, and methods and devices for driving the light-emitting element.

### BACKGROUND OF THE INVENTION

Neuroprosthetic intervention involves the recording of electrical activity from nerve cells and/or the inducement of new electrical activity in those cells. In so doing therapeutic benefit can be obtained. The very first commercialised neuroprosthesis was the heart pacemaker which was originally developed in the 1950's. This intervention has benefitted many by properly regulating the beating of the human heart. In the 1970's cochlear prosthetics were developed for the deaf. Such devices would pass auditory information from a microphone and pass it the brain by stimulating the cochlear nerve. Since then there have been developments in many areas including pacemakers for deep brain disorders such as Parkinson's disease, functional nerve stimulators to return motor function in the physically disabled, and visual prosthesis for the blind¹. ¹ Barrett John, Berlinguer-Palmini Rolando, Degenaar Patrick. Optogenetic approaches to retinal prosthesis. Visual Neuroscience 2014, 31(4-5), 345-354

The human nervous system consists of neuron cells which transmit information in an electrical manner within the cell and chemical manner between cells. The electrical model of the nerve cell was defined by Hodgkin and Huxley in 1956 {A. L Hodgkin, 1952 #38}². Electric activity is modulated by ionic flow in and out of cells, which is determined via chemically or electrically activated channels on the cell membrane. As such, the traditional method for recording and stimulating information flow in neurons has been with electrical means [1]. In particular, for the latter case, electrodes would pass charge (electrical current) into the ² A. L. Hodgkin and A. F. Huxeley A quantitative description of membrane current and its application to conduction and excitation in nerve. J. Physiol (1950) 117, 500-544 medium close to the cell and create a change in voltage across the cell membrane. This stimulus would activate electrically gated ion channels in the nerve cell membrane and would be followed by a reversal phase in the electrode to balance charge and prevent electrolysis.

In 2003, the ectopic expression of light-sensitive ion channels was first demonstrated [2]. This channel, known as channelrhodopsin-2 (ChR2) allows nerve cells to be stimulated with light of specific wavelengths. It has given rise to a field known as optogenetics—the genetic manipulation of cells to become photosensitive. In addition to ChR2, other photosensitive surface proteins are under investigation. Light sensitive ion pumps such as halorhodopsin {Zhao, 2015 #39}³ and light-activated protein amplifiers such as melanopsin [4, 5], and modified optically sensitive channels can provide a range of capabilities. These include both stimulus and inhibition from different wavelengths of light. These new tools have been revolutionary in the fields of electrophysiology and prosthetics [3-5]. As the sensitizing proteins can be genetically engineered into specific kinds of neurons, this technique can be fully used to explore complex brain circuits and neurological and psychiatric illnesses such as blindness, spinal cord injuries, Parkinson's disease and epilepsy [6-8]. Equally, this technique can potentially replace the use of electrical stimulus in medical neuroprosthetics, and the emerging field of bioelectronic medicine (also known as electroceuticals). ³ M. Zhao et al., “Optogenetic tools for modulating and probing the epileptic network,” Epilepsy Research, vol. 116, pp. 15-26, Oct, 2015

The key advantages of optogenetics over electric stimulus are multifold:
1. Very precise information coding can be achieved
2. The stimulus can be genetically targeted to specific cell types allowing much more precise control and information delivery. Electrical methods stimulate everything within the electric field profile.
3. Both stimulus and inhibition can be achieved by optical means, whereas electrical methods can only achieve stimulus.
4. It is possible to record simultaneously with the optical stimulus, whereas electrical stimulus creates large artefacts in the recording signal.

The key requirement in optogenetics is to deliver intense light (typically up to 1 mW/mm² in pulses of around 10 ms) locally to the nerve cells [2, 3]. The wavelength sensitivity peaks vary between different opsin proteins. The light sensitive core of these proteins—retinal, is the same as for the rhodopsin's used in mammalian vision. As such, their chemistries can be tuned across the visible range of the electromagnetic spectrum. The light requirement varies with both the opsin type and the efficiency of genetic expression on the cell membrane. At the time of writing the most commonly used wavelength is 470 nm (blue) for channelrhodopsin. However, shorter wavelengths, in particular, scatter strongly in tissue, requiring either extremely intense stimulus from an internal source or a local stimulus close to the nerve cell. The light requirement is a function of the irradiance and illumination time. Opsins have light and dark adapted states. So short radiance illumination is preferable⁴, though the efficiency of most light emitters decreases with intensity, so a trade-off is required between the two. ⁴ Grossman N, Nikolic K, Toumazou C, Degenaar P. Modeling Study of the Light Stimulation of a Neuron Cell With Channelrhodopsin-2 Mutants. IEEE Transactions on Biomedical Engineering 2011, 58(6), 1742-1751

In addition to optogenetic stimulus or neural activity, there is also increasing interest in optical probing of neurological function. Genetically encoding of reporter dyes such as gCAMP3 [6] can be used to measure cell activity directly. Furthermore, such dyes can be linked to calcium function and have thus potential in determining other functions in both neurological and non-neurological cells. Furthermore, in the non-optogenetic field, optical probing of cellular autofluorescence through fluorescence lifetime measurements and related techniques can report back useful information. Such techniques involve illuminating tissue in a similar fashion as per optogenetics and then recording spectrally or temporally shifted information using light recording techniques such as photodiodes. As the stimulus is typically on the bluer end of the spectrum [7], the same principles of light scattering apply, requiring the same principles of light delivery.

Given scattering effects in tissue, light may be delivered locally either through an optic guiding mechanism from afar [8] or via local generation on a penetrating probe [9]. The former is potentially convenient when very few individual stimulus points are required. Optical multiplexing is very challenging to incorporate into micro-sized penetrating probes. Probes have therefore been developed with multiple light guides [8] and multiple connection points. However, in this case, there is a complexity issue with the coupling of multiple individual light emitters with low losses, which is currently unsolved. The alternative approach is to develop probes which generate light locally in the tissue. Such devices are limited in intensity by thermal heating limits in tissue, but can still provide sufficient radiance to achieve suitable stimulus [10].

Light generation, either locally on the probe or by light guided methods can be achieved primarily by light emitting diodes [9, 11], lasers⁵, or by light emitting diodes with laser-like properties⁶ In all cases, the energy used to generate light will originate in electrical form, whether it comes from a battery, is scavenged in some form from the body, or transmitted percutaneously. As such, the typical configuration consists of an anode and cathode contact between the electrical circuit and the light emitter. It is additionally conceivable to have light emitting transistor structures⁷. ⁵ Hamaguchi, T., Fuutagawa, N., Izumi, S., Murayama, M., & Narui, H. (2016). Milliwatt-class GaN-based blue vertical-cavity surface-emitting lasers fabricated by epitaxial lateral overgrowth. Physica Status Solidi a-Applications and Materials Science, 213(5), 1170-1176. doi: 10.1002/pssa.201532759⁶ Hu, X. L., Liu, W. J., Weng, G. E., Zhang, J. Y., Lv, X. Q., Liang, M. M., . . . Zhang, B. P. (2012). Fabrication and Characterization of High-Quality Factor GaN-Based Resonant-Cavity Blue Light-Emitting Diodes. Ieee Photonics Technology Letters, 24 (17), 1472-1474. doi: 10.1109/Ipt.2012.2206110⁷ Raffaella Capelli, Stefano Toffanin, Gianluca Generali, Hakan Usta, Antonio Facchetti & Michele Muccini “Organic light-emitting transistors with an efficiency that outperforms the equivalent light-emitting diodes” Nature Materials 9, 496-503 (2010)

The emitter, whether in laser, laser-like or light emitting diode form, is typically constructed from an emissive semiconductor layer sandwiched between conductive structures which respectively provide the electrons (cathode) and holes (anode). The semiconductor layer may be constructed from organic substrates⁸, inorganic crystal⁹ or quantum dots¹⁰. ⁸ Raffaella Capelli, Stefano Toffanin, Gianluca Generali, Hakan Usta, Antonio Facchetti & Michele Muccini “Organic light-emitting transistors with an efficiency that outperforms the equivalent light-emitting diodes” Nature Materials 9, 496-503 (2010)

Liu, J., Zhang, H. T., Dong, H. L., Meng, L. Q., Jiang, L. F., Jiang, L., . Heeger, A. J. (2015). High mobility emissive organic semiconductor. Nature Communications, 6, 8. doi: 10.1038/ncomms10032 ⁹ Maaskant, P. P., et al., High-Speed Substrate-Emitting Micro-Light-Emitting Diodes for Applications Requiring High Radiance. Applied Physics Express, 2013. 6 (2).¹⁰ P. Anikeeva; J. Halpert; M. Bawendi; V. Bulovic (2009). “Quantum dot light-emitting deices with electroluminescence tunable over the entire visible spectrum”. Nano Letters. 9 (7): 2532-2536Seth Coe; Wing-Keung Woo; Moungi Bawendi; Vladimir Bulovic (2002). “Electroluminescence from single monolayers of nanocrystals in molecular organic devices”. Nature. 420 (6917): 800-803.

Optogenetic illumination requires ultra-bright emission, which may be beyond the best organic light emitting diode (OLED) structures currently be produced¹¹. The same may be true for quantum dots. Additionally, the operational lifetime of OLEDs decreases inversely with the operational current density. As the drive current density is directly proportional to the LED area and emitter radiance, their suitability for optogenetics is currently limited. However, as the molecular biology improves and the light requirement is reduced, they may prove to be useful tools in the future as their emission wavelength peaks are broadly tuneable through chemistry from violet through to the near infra-red¹². ¹¹ Liu, J., Zhang, H. T., Dong, H. L., Meng, L. Q., Jiang, L. F., Jiang, L., . . . Heeger, A. J. (2015). High mobility emissive organic semiconductor. Nature Communications, 6, 8. doi: 10.1038/ncomms10032¹² Soultati, A., Papadimitropoulos, G., Davazoglou, D., Argitis, P., Alexandropoulos, D., Politi, C. T., . . . Ieee. (2015). Near-IR Organic Light Emitting Diodes Based on Porphyrin Compounds 2015 17th International Conference on Transparent Optical Networks

The same principle holds true for quantum dot technologies whose emission spectra are tuneable through the size of the quantum dot. However, quantum dots typically contain cadmium or lead which is toxic if leached out into the body through degradation. Acute and short-term studies of encapsulated quantum dots in animals has been performed without ill-effect. However, long-term studies indicate significant toxicity¹³. ¹³ Cao, Y. H., Wang, D., Li, Q. Z., Deng, H. L., Shen, J., Zheng, G. Y., & Sun, M. (2016). Rat Testis Damage Caused by Lead Sulfide Nanoparticles After Oral Exposure. Journal of Nanoscience and Nanotechnology, 16(3), 2378-2383. doi: 10.1166/jnn.2016.10938

Inorganic light emitting diode materials such as Gallium Nitride (GaN: violet to green emission) and Aluminium Gallium Indium Phosphide (AlGaInP) allow for much higher current densities. In particular, GaN can be tuned within the 450 nm-510 nm range which matches the most common opsin absorption peak of 470 nm. They can be fabricated into microscale dimensions and are constructed with multiple quantum well layers for efficiency. Crucially for implantable devices, their operational lifetimes are long, even for relatively high current densities, and there is no current evidence of cytotoxicity from degradation products. Thus, they are a primary candidate for high-radiance optogenetic stimulation are gallium nitride based LEDs [14, 15].

For sensing applications, including optogenetic sensing, lower light intensities may be acceptable, allowing organic and quantum dot emissive technologies. Furthermore, in some cases, the primary wavelength of interest may be in the infra-red. In such cases, Aluminium Gallium Arsenide, which is used extensively in the telecoms sector can be applied.

In the case of local light generation on the probe, the optical emitter will be near the tissue interface, and therefore there is the possibility of electrochemical degradation. In the case of light guided probes, if the optical emitter is potentially further away from the tissue and under greater encapsulation. However, it may still be susceptible to degradation. Therefore, this application is directed towards ways in which electrochemical degradation of anode and cathode contacts in implantable optical emitters may be prevented or reduced.

### Degradation Mechanisms

Before turning to the invention in detail, it is useful to understand the mechanisms by which degradation of anode and cathode contacts may occur.

Electrochemical degradation is a well-known and understood phenomenon. If a metal is placed in a saline solution, electric fields between the metal and saline will induce ion transfer. This transfer will result in the degradation of the metal in the interface with the solution. Wang, Q., Zhou, Y. F., Song, B., Zhong, Y. L., Wu, S. C., Cui, R. R., . . . He, Y. (2016). Linking Subcellular Disturbance to Physiological Behavior and Toxicity Induced by Quantum Dots in Caenorhabditis elegans. Small, 12(23), 3143-3154. doi: 10.1002/smll.201600766

In the case of implantable electronic devices, electrodes typically have circuits and capacitive structures which try ensure that any electrical interaction with the body has no net charge i.e. direct current delivery. Rather, electrode stimulus is presented in an alternating current (AC) fashion with any positive charge pulse matched by a negative charge pulse. The integral function of the two pulses needs to be zero.

However, electrode structures are surface facing and thus in direct contact with the tissue fluid. Other structures will have passivation layers (e.g. silicon dioxide or silicon nitride) and are typically encapsulated with a polymer such as silicone or parylene. In other configurations, there may only be a ceramic passivation layer or a polymer coating. Their degradation mechanism is less obvious as there should be no electrochemical interface. Most polymers can allow for limited aqueous transmission. Therefore, any cavity caused by defects in the interface between the passivation material and the active layers will attract and be filled with water and/or saline. Such defects may be inherent in particular manufacturing processes. Alternatively, they may be created over time due to delamination between layers or through aging-related stresses or chemical changes in the materials.

The creation of saline filled cavities can start electrochemical degradation in neighbouring structures if there are electric fields. Typically, electrical lines controlling electrodes, and surface photonic structures will be placed in a dielectric stack near the surface as per FIG. 5. These will result in electric field distributions described in FIG. 6. Such fields in the presence of cavities—whether through defect or ageing, can cause a degradation over time leading to eventual failure.

Furthermore, there is a process whereby electrochemical exchange with a fluid filled cavity can cause expansion of the cavity and thus further delamination. Such processes can expand a failure point from a very localized region to the whole implant. The degradation process is very nonlinear, so difficult to determine and predict. However, in more generalised terms, the process will be hastened by the strength of net electric fields, and the temporal duration of the field. Implantable structures need to be small. Thus, the distance between electrically active structures is small, which in turn increases electric field strength (=voltage/distance). Optogenetic stimulation is typically in the 1-100 millisecond range to provide sufficient activation. Though short, significant degradation processes may occur at this time. These effects are clearly undesirable, particularly in implants which are placed in the brain and are difficult to explant and replace.

Methods are therefore required to reduce such degradation mechanisms. These include better manufacturing techniques to prevent voids, choice of materials, and design of electrical lines with sufficient spacing to reduce electric field strengths.

In contrast to electrodes, light emitting structures have a strongly rectified current-voltage operation. Thus it is not feasible to balance charge over time. However, such currents are not actually in contact with the solution. So the mechanism of degradation is quite different to electrode driving. Rather it is the electric fields which may cause ionic flows between electrodes and any fluid filled cavities. As such, the principle is to balance electric fields on the cathode and anode with respect to time.

### Background Art

Numerous illumination systems exist in industry. Increasingly organic light emitting diodes (OLEDs) are being utilised in displays for both emplaced and mobile systems. Although OLEDs have the potential for beautiful quality displays, it has been notoriously difficult to ensure operation in years rather than months. As such, much effort has been placed on exploring methods to ensure their long-term lifetime.

For consideration with regards this invention, researchers in this field have considered electronic biasing techniques to minimize long term degradation. Eisenbrand et al. [12] have considered separating sub-frame pulse width intensities to reduce current voltage stresses. But their proposed degradation mechanism is related to current density and [current×time] rather than through electrochemical mechanisms. Other researchers in the OLED field have considered biphasic operation.

Lin et al. [13] considered the problem of long-term threshold voltage Vₜₕ change in the organic diodes. Simply increasing the supply over time steadily increases the rate of degradation and thus the operational lifetime of the device. They thus considered an alternative positive/negative biasing scheme to reduce the effect of Vₜₕ change without hastening the electric field induced degradation.

Such reported biphasic operation in the driving of OLEDs is operating on a fundamentally different principle to the electrochemical mechanism proposed in this invention. Furthermore, their implementation is a three transistor circuit within a matrix scheme with a global positive/negative bias. In our case, not only is the fundamental physics of the degradation effect different, but we are considering both the effects of differential electric fields between cathode and anode and common fields between cathode/anode and the tissue. As such, the circuitry we propose is very different.

Previously, optical systems for retinal prosthesis have been demonstrated [11, 16], but in this configuration, the illumination system is external to the body. As such, the control transistor is simply implemented to provide maximum power per pixel and modulated in time. Alternatives could be to modulate with current, but it is not necessary to consider voltage balancing. Others have also considered high power light emitting diode arrays for illumination purposes [14, 17]. However, in these cases, although the circuitry is not presented, there is also no requirement for voltage balancing during operation, and such operation is not mentioned in the text.

Some groups, have considered implantable probes incorporating LEDs [9, 10, 18]. Some such as McAlinden et al. [9] have created probes directly from the Gallium Nitride. Others such as Cao et al., and Clements et al. [19, 20] have developed silicon-based probes with embedded Gallium Nitride LEDs. However, most of these groups have used discrete current drivers and microcontrollers to drive the current. Past work by the Degenaar team has described microelectronic drive circuits in the form of a H-tree circuit which can provide current steering approaches which can use the reverse current for diagnostic purposes [18]. However, electric field balancing [voltage×time] for long-term degradation prevention has not previously been demonstrated.

### SUMMARY OF THE INVENTION

In order to prolong the lifespan of the light-emitting element by prevention of degradation caused as a result of net electric fields, the present invention provides at its most general, an optical stimulation arrangement in which a light-emitting device is driven in a manner which balances out the voltage-time profiles in a biphasic manner, in order to invert any charge which has built up in its surrounding environment. In other words, structures with potential exposure to aqueous fluid need to obey two rules:
1. The integral function of electric field oscillations between any two structures (i.e. differential mode fields) with potential exposure to fluids should be zero.
2. The integral function of the electric field between any structure and the surrounding environment should be zero.

As microelectronic structures can have placements much closer to each other than with the common tissue, rule 1 is in many cases most significant. Additionally, as microphotonic (or similar sensor/actuator) structures are bonded onto the surface of microfabricated probes, the most significant effect will be on the anode and cathode contact of the microphotonic device. Where there is a plurality of contacts, for example in a 3 contact light emitting transistor, the principle can simply be extended for all contacts. Furthermore, the control lines which connect to these contacts are also susceptible to degradation and should where possible operate under the same principles.

These principles hold true for other electrically active devices that are embedded on implantable probes. These may include Micro Electro Mechanical Systems (MEMS), microbolometer or solid state thermal sensors, piezo actuators, memristors, antennae and rectennae. These have different operational voltage properties, but the mechanism by which they may degrade through electric field induced electrochemical degradation is the same. For brevity, we describe the specific microphotonic case, but the inventive circuitry holds true for all.

Accordingly, a first aspect of the present invention provides an optical stimulation arrangement including:
a light-emitting device, implantable in an environment with an associated ground voltage, the light emitting device including:
   a light emitting element;
   an anode; and
   a cathode; and
a controller for driving the light-emitting device in a biphasic manner.

Optional features of the invention will now be set out. These are applicable singly or in any combination with any aspect of the invention.

Preferred embodiments of this invention are directed towards optical stimulation of tissue, and preferably neural tissue such as brain tissue or retinal tissue. There is also a growing body of work in the field of bioelectronics medicine (also known as electroceuticals) which may require such devices to be placed in non-neural tissue. In these cases, the surrounding environment is the tissue itself and the associated ground voltage is referred to as “the tissue ground” or “gndₜᵢₛₛᵤₑ”. Stimulation of neural tissue has important applications for optogenetic techniques, as described earlier in this application.

In preferred embodiments of the present invention, the light-emitting element is a semiconductor light-emitting element such as a light-emitting diode (LED), a laser, or a light emitting diode with partial laser-like qualities. The operational physics of these devices is different, but they have similar current-voltage characteristics, which is important for the present invention. The most important consideration is the rectification properties which will determine the stimulation and reversal driving profiles.

The light emitting element may be constructed from organic semiconductor material such as 2,6-diphenylanthracene (DPA), inorganic crystalline materials such as gallium nitride (GaN), or quantum dots such as CdSe/ZnSe. Generally, for Implantable devices there is a requirement for high radiance stable devices with non-toxic byproducts. GaN therefore can provide the primary wavelength of 470 nm for some opsins, and AlGaInP for the 590 nm peak. Alternative materials can be used for other requirements including sensing requirements.

The spectral range required for stimulating different opsin forms ranges from 450 nm-650 nm. For sensing applications, this range may extend to the infra-red.

The anode and cathode of the light-emitting device may be connected directly to a power supply, which may be included in the optical stimulation arrangement. Alternatively, the optical stimulation arrangement may be connectable to an external power supply, such as a battery. Specifically, the biphasic driving of the light-emitting device is achieved by controlling the voltage inputs at the anode and the cathode. The controller may be configured to drive the light-emitting device with a stimulation phase and a reversal phase, each having an associated voltage-time profile, and preferably wherein the voltage-time profile associated with the reversal phase is selected to balance out the voltage-time profile associated with the stimulation phase. In this way, net electrochemical oxidation/reduction (or addition or subtraction of material) from the anode/cathode with respect to an adjacent aqueous layers will be reduced. The lifetime of the anode/cathode and/or the control lines is thus prolonged, thus prolonging the life of the light-emitting device. As the name suggests, the light emitting element is preferably in an ON state during the stimulation phase. Ideal diodes have zero current under reverse bias. However, in practice, there will be a leakage current defined by the rectification properties of the diode. Typically, this is in the nano-amps range and is non-radiative. So, in preferred embodiments, the light-emitting element is in an OFF state during the discharge phase.

The voltage-time profiles associated with the stimulation and reversal phases may be selected to balance out by selecting the reversal phase so that the integral of its voltage-time profile is equal or substantially equal to the negative of the integral of the voltage-time profile associated with the stimulation phase. In this way the charge built up as a result of the electric fields associated with the applied voltages sums to zero, or substantially zero, reducing the extent of any degradation.

Some electrochemical degradation is irreversible, but such processes also have time constants. So it is advantageous to modify the stimulation and reversal times to be short. In the preferred embodiment ON illumination times are in the range of 1-100 ms (but may be 1 microsecond to 100 ms). However, the total illumination time can be interleaved with multiple stimulation and reversal phases. As such, the integral function of the stimulation remains the same for a single longer pulses. However, by utilising higher speed interleaving, whereby the pulse width of the stimulation phase is 1-1000 microsecond, a further reduction in degradation can be achieved. In such cases there may need to be an increase in the total integral illumination to compensate for opto-neural adaptation effects beyond 10 ms.

In preferred embodiments, the controller may be configured to drive the light-emitting device in a third state, referred to herein as a neutral phase. In this state, the voltage across the anode and the cathode are the same as the ground level of the surrounding environment. In preferred embodiments, during the neutral phase, the voltage is clamped to the tissue ground level. It is envisaged that the controller causes the light-emitting device to be in the neutral phase between periods when optical stimulation of the environment is required. In this state, since the anode and the cathode are both fixed at the ground voltage, there is no differential voltage difference between the two, nor is there a voltage between them and the surrounding environment, and accordingly, no electric fields arise, and degradation is minimized.

In order to switch between the two or three phases as described above, the controller preferably includes control circuitry configured to switch between the phases. In particular, the control circuitry preferably includes a switching arrangement configured to switch between all of the phases. For example, in embodiments where there are only two phases, it is preferable that the control circuitry is switchable between two configurations (one in which the stimulation phase is selected, and one in which the reversal phase is selected). The same applies for the preferred embodiments which further cater for a neutral phase (i.e. a third configuration is available). The control circuitry preferably includes at least one switch in order to effect switching between the various phases. Furthermore, the control circuitry preferably also includes a current source. In preferred embodiments, one or both of the at least one switch and the current source may be implemented using one or more transistors. More details of this implementation are set out in the “Detailed Description” section. Transistors may also be used to ensure clamping to the ground level (e.g. tissue ground) in the neutral phase. Furthermore, in order to ascertain the ground voltage to which the voltage across the anode/cathode must be matched, the control circuitry (or another part of the controller) preferably includes means for measuring the associated ground voltage. This means may also be implemented in the form of a transistor, as is explained in detail later on in the application.

As has been discussed previously, a preferred application for optical stimulation arrangements according to the present invention is in the stimulation of body tissues, particularly neural tissue. Accordingly, the light-emitting device, which may be in the form of an optrode, or a plurality of optrodes. The optrode(s) are preferably mounted on a mounting plate, the arrangement of optrode(s) and mounting plate forming an implant which is implantable into the tissue of a user. The tissue is preferably neural tissue. In such configurations, the controller is preferably separate from the light emitting device, but connected to it, e.g. via a connective lead. The controller may be contained in a central control unit connected to the light-emitting device via connective leads or via a wireless connection. The central control unit may also include the above-mentioned power supply, e.g. a battery pack. Alternatively, the central control unit may be configured to receive a battery. In use, the central control unit may also be implanted into the user, though not necessarily in the same place as the light-emitting element. For example, as is discussed later, the central control unit may be implanted in the chest.

In order to communicate with devices external to the optical stimulation arrangement of the present invention, the controller may include means for wirelessly communicating with external electronic devices, e.g. for control purposes. This communication may, for example, be in the form of Bluetooth or VVi-Fi communication. The wireless communication preferably falls within accepted ISM or Medradio bands.

A second aspect of the present invention provides a controller configured to drive a light-emitting device in a biphasic manner, the light-emitting device being implantable in an environment with an associated ground voltage. All of the optional features presented above may also be combined with embodiments of the second aspect of the invention, where compatible.

A third aspect of the present invention provides a method for driving a light-emitting device in a biphasic manner. As with the second aspect of the invention, the optional features which are set out with respect to the first aspect apply equally to the third aspect, where applicable.

Further optional features of the invention are set out below, described in detail with respect to the attached drawings. These optional features, again, are compatible with all aspects of the invention.

### BRIEF DESCRIPTION OF THE DRAWINGS

Embodiments of the invention will now be described by way of example with reference to the accompanying drawings, summarized briefly below.

FIG. 1: The Neuroprosthetic concept. A central control unit provides power and control to the interface unit in addition to communication with the outside world. Both units and the lead between are implanted subcutaneously.

FIG. 2: The Global power system. The power transfer from the central control unit to the implant unit is in alternating current format. AC is to ensure no damaging DC current leak into the tissue in the case of a cable break. An AC to DC converter then reconverts this back to a DC supply at the optrode, providing V_{dd} and Vₛₛ supply lines

FIG. 3: Optrode variants. (a) Neuroprosthetic interfaces to nerve bundles require a wrap-around format known as a cuff optrode. (b) Planar devices can be suitable for brain surface or retinal stimulation. (c) For brain stimulation, an implantable probe is required which may be limited to the cortical regions, or alternatively, be utilised to penetrate to deeper regions such as the thalamus or subthalamic nucleus. (d) For cortical interfaces, an array of penetrating probes, typically 2-6 mm in length can be used to record from and/or stimulate designated areas.

FIG. 4: Intelligent optrode concept. A communication unit can receive commands from the central control unit and return data such as diagnostics. It is typically formed as a finite state machine and can have internal command sequences to implement sequential timed operations. The power supply provides sources for the digital and analog electronics as well as the photonic emitters. The typical operation will consist of recording units, stimulation units, and diagnostic units

FIG. 5: Micro-emitter contact site. Typically, electrical lines controlling electrodes, and surface photonic structures will be placed in a dielectric stack near the surface. An anode and cathode contact are then presented to the emitter which is typically a light emitting diode (LED). The combined structure is then covered with a passivation material, typically comprising of a polymer such as parylyene or silicone.

FIG. 6: Degradation mechanisms. The electric fields between cathode and anode, between electrical lines and between anode/cathode and tissue ground.

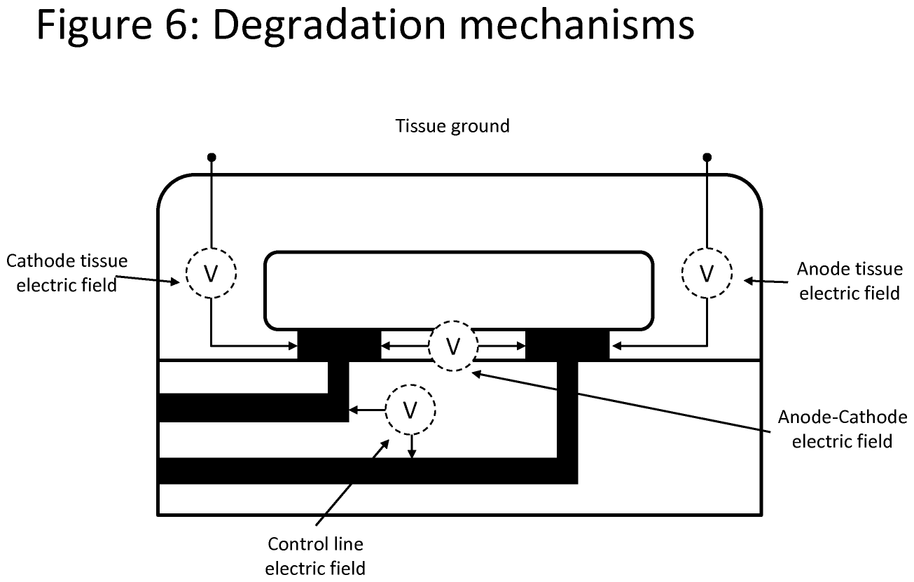

FIG. 7: Photonic operation of a gallium nitride micro-light emitting diode. A strong rectification can be seen between the forward and reverse voltage drive—mA vs. nA. The curve also demonstrates light emission which only occurs in the forward voltage domain.

FIG. 8: Photonic control waveforms. Light emission from micro-emitters can follow five operational sequences: (Phase 1) LED OFF, Cathode/Anode at ground. (Phase 2) LED ONAnode V+, Cathode V− ground. (Phase 3) LED OFF, Cathode/Anode at ground. (Phase 4) LED REVERSE, Anode V−, Cathode V+ ground. (Phase 5) LED OFF, Cathode/Anode at ground. (a) describes a typical LED operation without [voltage×time] balancing or clamping to ground. (b) describes a symmetrical biphasic [voltage x time] balancing, and (c) describes an asymmetric approach.

FIG. 9: Photonic interleaving. The total optical stimulus on either the optogenetically encoded neuron or sensor fluorophore will be an integral function of the irradiance on the cell with respect to time. As such the effective intensity can be modulated by either radiance or emission time. In order to reduce degradation, the stimulation/reversal cycle time can be reduced. In this case, it needs to be repeated multiple times such that the total ON time presented in (b) is the same for that presented in (a).

FIG. 10: Stimulation artefacts—mechanism

FIG. 11: Stimulation artefacts effect

FIG. 12: Reducing stimulus arteface

FIG. 13: Reducing stimulus arteface

FIG. 14: pseudo code

FIG. 15: combined waveform

FIG. 16: LED pulse control. A current source determines the level of current flow and thus light output in the ON state. Switches then allow for three operational phases: In the neutral phase (a) there is no voltage across the diode and thus no current flow. The common-mode potential of the anode and cathode is also set to tissue ground. In the stimulation phase (b), current flows in the forward direction through the diode allowing light emission. In our proposed embodiment, the anode potential would be greater than the tissue ground and the cathode potential would be less. In the reversal phase (c), there is typically a very small current flow in the reverse direction, depending on the rectification properties of the diode. In our proposed embodiment, for this phase, the anode potential would be less than the tissue ground and the cathode potential would be greater.

FIG. 17: Biphasic control circuit description. The image shows a schematic of the control circuit which can be designed in a (Complementary Metal Oxide Semiconductor) CMOS process. M1-M4 represent transistors which are used to determine the direction of current flow through the diode. M1 and M2 also act as analog transconductance amplifiers to determine the amount of current flow through the diode, and thus the emitted light intensity. The current flow is controlled by the digital to analog converter and ‘AMP’, which represents an inverting voltage amplifier. M5-M8 allow clamping the anode and cathode to tissue ground. These can be included if the resting potential of the anode and cathode without such structures deviates from tissue ground. This is typically the case if M1-M4 are not the same size. If the deviation from tissue ground is small, M6 and M7 can be negated. Additionally, if a triple well or SOI CMOS process can be used with an isolated n-well, then the body of M5 and M8 should be connected to tissue ground to prevent a body effect. Switches S1 to S10 represent digital switches which can be implemented as transistors. For some ‘AMP’ implementations, it may be difficult to achieve the maximum values of V_{dd} and Vₛₛ to correspond with the DAC values 0 and 255. Thus, S₁ and S₄, can be used to clamp to V_{dd}, and S₃ and S₆ to clamp to Vₛₛ.

FIG. 18: Control logic configuration. The Logic states used for operating the circuit described in FIG. 10. For some amplifier embodiments, the maximum DAC value may not result in maximum current drive. Additionally, some embodiments may be driven entirely with pulse width modulation. So ‘full’ represents maximum driving driving current in either stimulation or reversal phase.

FIG. 19: Optrode embodiment. The optrode is manufactured from a complementary metal oxide semiconductor (CMOS) substrate. Gallium nitride micro-LEDs with a centre surround format have been bonded on-top. In this configuration, the anode is in the centre and the surround is the cathode. The inset image describes the illumination thereof

FIG. 20: Circuit embodiment. The computer aided design layout of the emitter control circuit and biphasic circuit in 0.35 μm CMOS technology.

FIG. 21: Simulation results of stimulation circuit. The simulation results achieved for the driver circuit in combination with a light emitting diode. The circuit can provide a large output voltage (up to 4.5V) and current (up to 6 mA) across the LED anode-cathode, thus ensuring high radiance emission from the photonic emitter

FIG. 22: Simulation results of light emitting diode biphasic operation. (a) for driving 1 mA through our circuit balancing 3.4×10⁻³ Vs (b) for driving 3.4 mA, and balancing 4.7×10⁻³vs

### DETAILED DESCRIPTION AND FURTHER OPTIONAL FEATURES OF THE INVENTION

In addition to the description of the embodiments set out below, further detailed description of the embodiments is set out in Annex A, titled “Biphasic Micro-LED Driver for Optogenetics”.

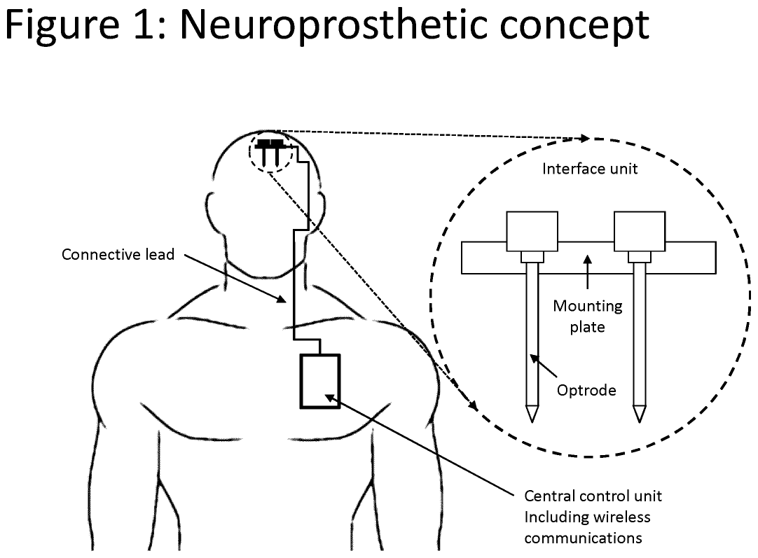

FIG. 1 describes a typical neuroprosthetic implantable system. A central control unit provides power and control to the interface unit in addition to communication with the outside world. Both units and the lead between may be implanted subcutaneously. As discussed, in alternate configurations, the control unit may be implanted in areas other than the chest, such as on the skull, or lower abdomen. Additionally, some sensory prosthetics such as visual and auditory prostheses may not have a battery unit, but instead receive continuous power and communications from an external source.

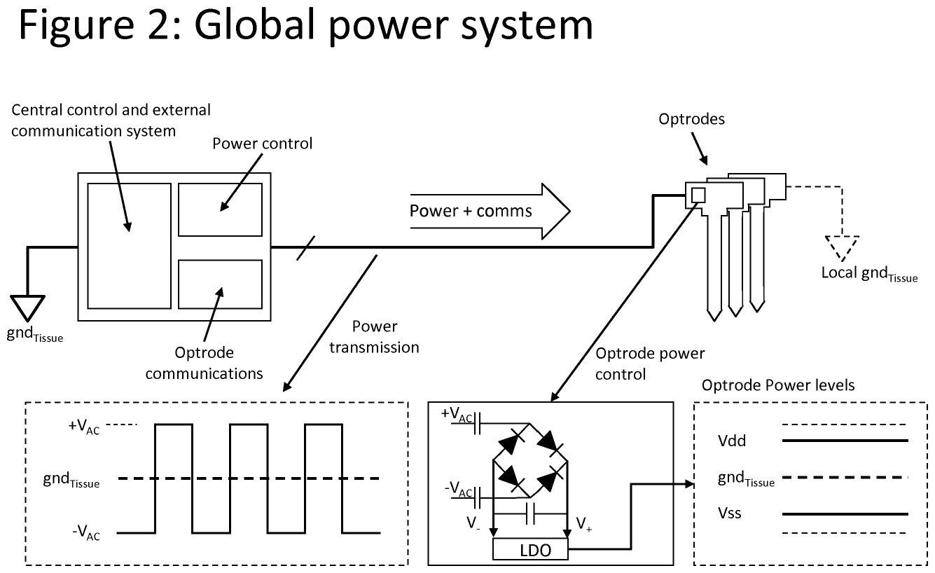

FIG. 2 describes the power transfer from the central control unit to the implant unit with implantable probes (optrodes). The power is converted from DC to AC to prevent a damaging net direct current leak into the tissue in the case of a cable break. An AC to DC converter then reconverts this back to a DC supply at the optrode, providing V_{dd} and Vₛₛ supply lines. In practice, there may be more than one V_{dd} and even Vₛₛ line as the voltage required for blue LED operation is typically up to 5V, whereas modern transistor logic is in the range of 1-3.3V depending on the technology node. However, for the sake of brevity, we describe here simply V_{dd} and Vₛₛ. The oscillating AC signal should oscillate around the tissue ground point, which should then also be at the midpoint between V_{dd} and Vₛₛ.

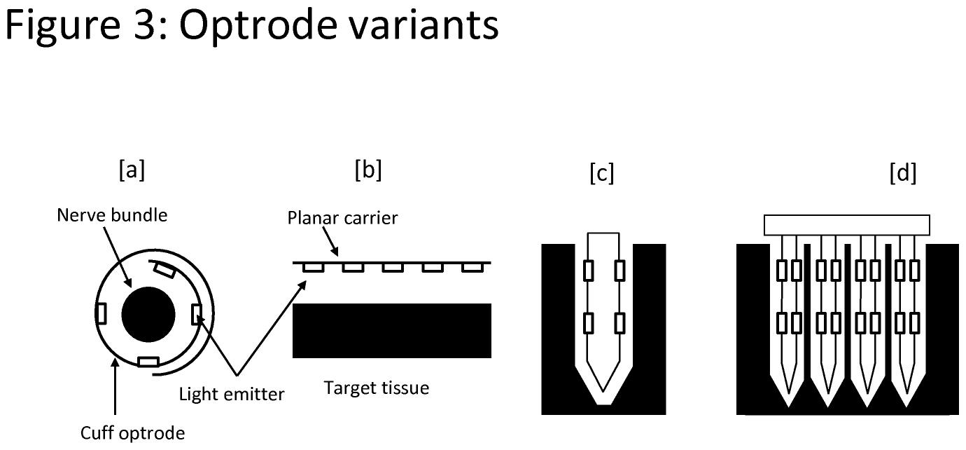

There are some different configurations for the interface unit, which are described in FIG. 3. Neuroprosthetic interfaces to nerve bundles require a wrap-around format known as a cuff optrode. Planar devices can be suitable for brain surface or retinal stimulation. For brain stimulation, an implantable probe is required which may be limited to the cortical regions or alternatively, be utilised to penetrate to deeper regions such as the thalamus or subthalamic nucleus. For cortical interfaces, an array of penetrating probes, typically 2-6 mm in length can be used to record from and/or stimulate designated areas.

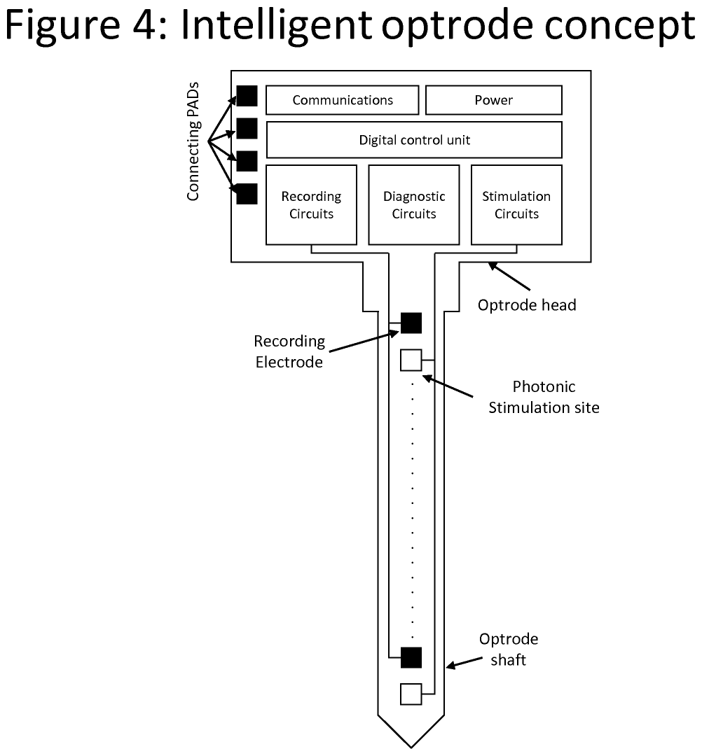

Multiple independent probes incorporating multiple independent illumination elements require internal microelectronics for control and multiplexing. Such electronics are described in FIG. 4. A communication unit can receive commands from the central control unit and return data such as diagnostics. It is typically formed as a finite state machine and can have internal command sequences to implement sequential timed operations. The power supply provides sources for the digital and analogue electronics as well as the photonic emitters. Though typically at different voltages, these have been described in aggregate as V_{dd} and Vₛₛ. The typical operation will consist of recording units, stimulation units, and diagnostic units [18]. On one configuration, the optrode shape may be cut out from microelectronic circuitry. Alternatively, a microelectronic head may be bonded on to an existing optrode. In the former case, it may prove to be more compact to incorporate circuitry in the shaft of the optrode.

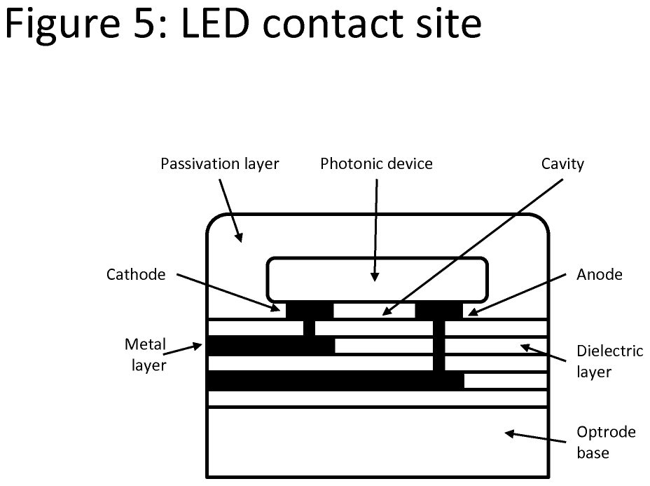

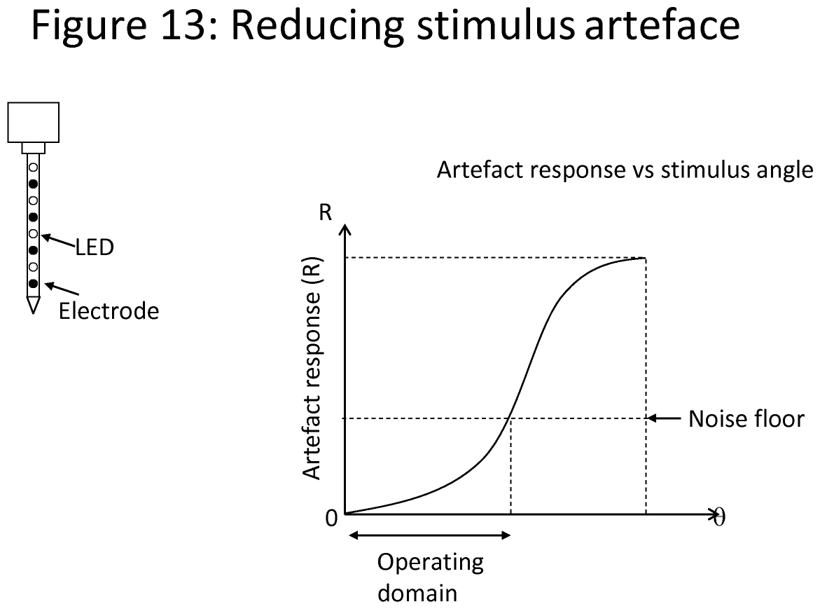

FIG. 5 shows the bonding arrangement of a micro-emitter onto the optrode. The emitter in this embodiment is a light emitting diode, but it may also comprise a laser or a diode with some laser properties. For all sole-emitters, bonding would be with an anode and a cathode. It may also be feasible to have a light emitting transistor structure which may have three or four connections, but this does not affect the main principle of operation. The geometric configuration of anode and cathode may be planar or in centre-surround fashion. I.e., the cathode may surround a central anode in a circular fashion which can be seen in FIG. 13.

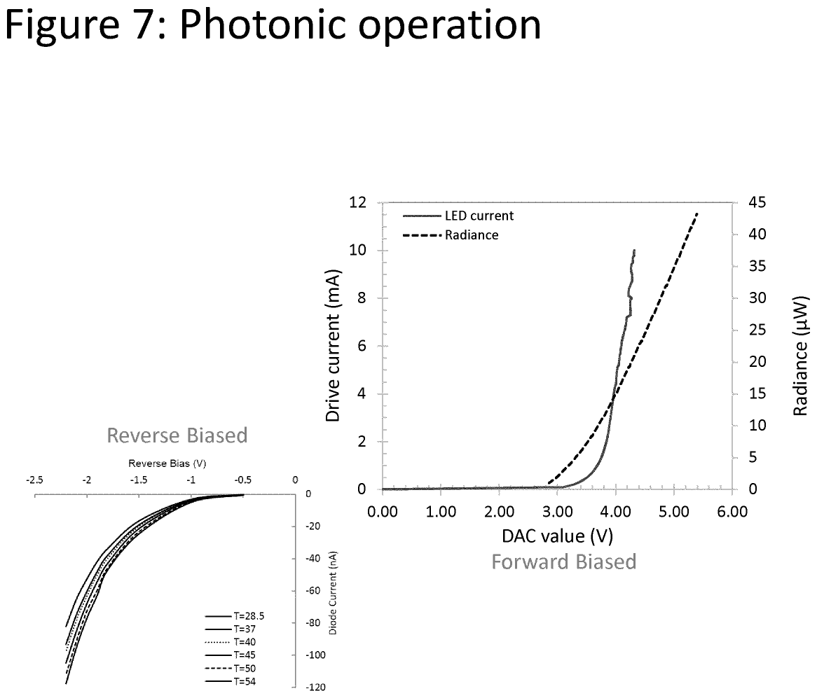

FIG. 7 describes the operation of a typical Gallium Nitride light-emitting diode with dimensions suitable for implantation. Micro-sized laser diodes would have similar electro-optical behaviour, albeit typically require much higher drive currents. Such diodes are constructed from multiple quantum wells and are fundamentally designed to trap electrons and holes and force recombination to produce photons. Their current-voltage relationship is exponential and limited by device and circuit resistances. There is also a strong rectification between the forward and reverse voltage drive as can be seen by the forward and reverse current levels, which is influenced by temperature (T28.5-T50° C.). Light is only emitted in the forward biased voltage domain.

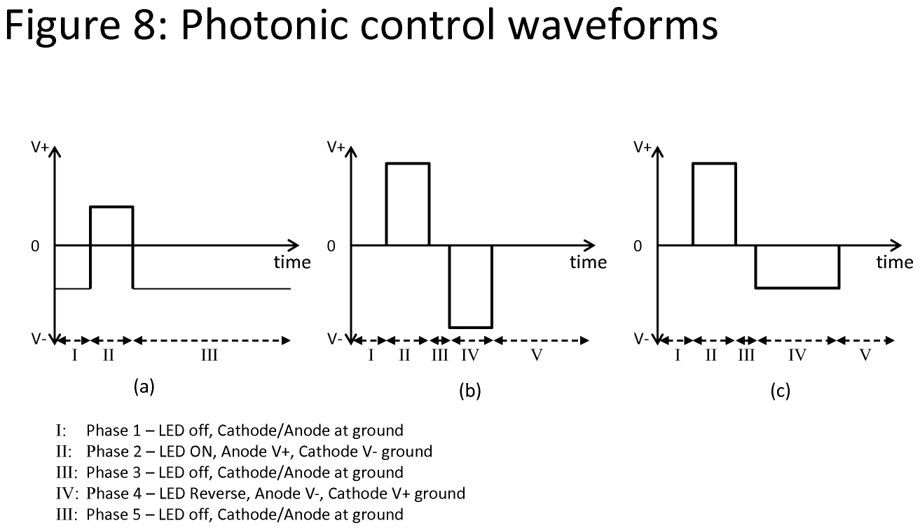

The typical operation of a light emitter from the perspective of anode voltage is given in FIG. 8(a). There are two effective states corresponding to the stimulation phase and the neutral phase. For much of the time, the emitter will be in the neutral phase. At this time no significant current is no light emission, and only small leakage current flow. Any leakage currents should be small, so the differential voltage across the anode and cathode should be small. However, as the anode/cathode will be close to Vₛₛ, there will be a net common voltage with respect to the tissue, assuming that Vₛₛ is different to the tissue ground point. During the stimulation phase, light will be emitted, and there will be a differential voltage across the anode and cathode, as well as a common voltage difference with the tissue medium.

The traditional operation, described above, results in both net electrode fields common to the cathode/anode and the tissue, and differential between cathode and anode. Resultant net electric fields can cause electrochemical degradation in the long term should defects create cavities which then fill with saline. As such, to reduce these effects, both the differential fields between anode and cathode and common field with the tissue need to be reduced, preferably to zero.

FIG. 8(b) describes operation according to an embodiment of the present invention. Initially, the emitter is in the neutral phase state, and both the differential and common voltages are fixed at zero. Then for the light to be switched ON, there is a forward voltage whereby the anode potential is brought towards V_{dd} and the cathode potential brought towards Vₛₛ, i.e. the light-emitting device is driven in the stimulation phase. The emitter is brought back to the neutral phase, i.e. the LED switches off, and then to the reversal phase. Here the cathode and anode voltages are reversed relative to the tissue ground. Then the system is brought back to the neutral phase, the LED remaining OFF.

The principle of operation is analogous to electrode charge balancing except that in this case, it is the electric fields with needs to be balanced. Thus the integral of the voltage with time should be balanced by the integral of the negative voltage over a given time. FIG. 8(b) describes how the reversal state may balance differences in voltage driving in each direction through the use of a longer (or shorter) time period.

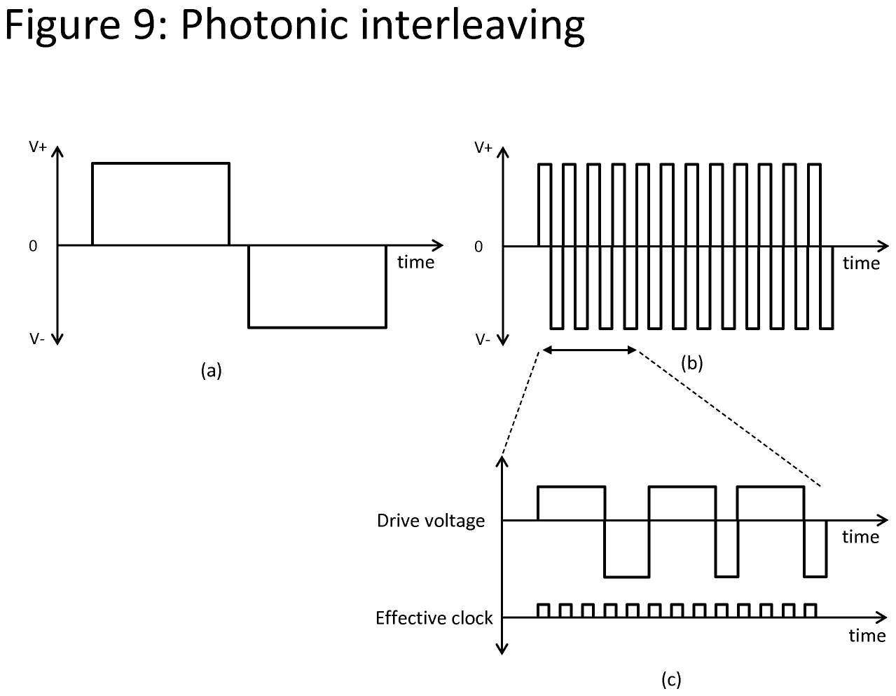

The total optical stimulus will be an integral function of the irradiance on the cell with respect to time. There is a limit to the maximum light output of LEDs, so typical stimulus times or in the period of 1-100 ms. However, longer periods of net electric fields increase the possibility of non-reversible electrochemical activity at the anode/cathode-fluid interface. As such, the stimulation/reversal cycle time can be shortened and repeated such that the total stimulation time is the same as for a single, longer stimulation/reversal cycle. This concept is presented in FIG. 9. The integral ON time for (a) and (b) are the same in this case. In practice, there will be some adaptation effects for optogenetically encoded neurons when stimulated over longer time scales, so this will need to be compensated with longer ON times for (b). As shorter cycling is better, stimulation reversal cycle times may be set to 1-1000 μs.

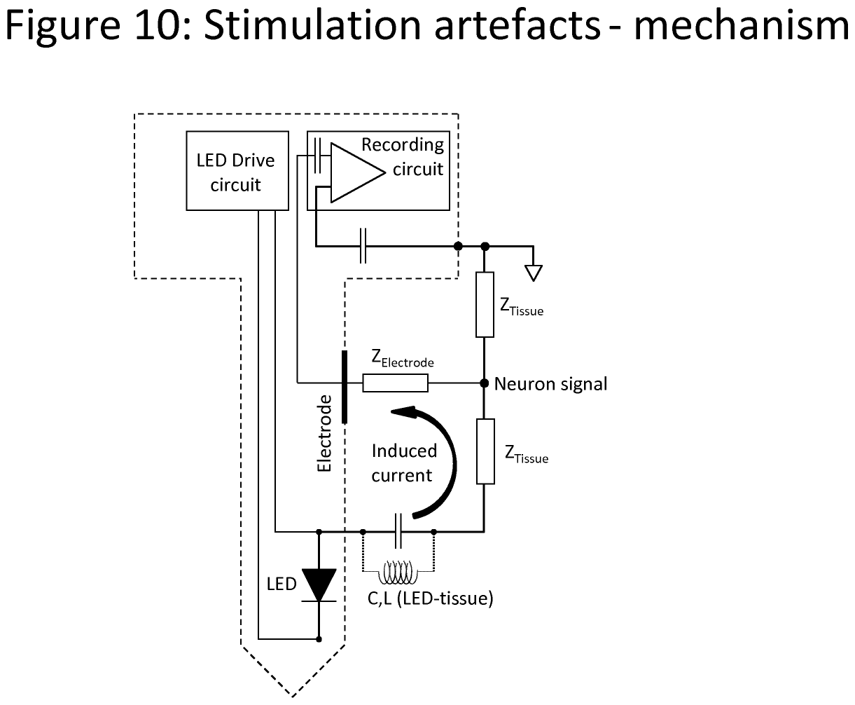

So far, the simple case of pulse modulation has been explained. However, there is a further issue with regards stimulus artefact. FIG. 10 describes a version of FIG. 4 where the electrical circuit with the neural tissue is considered. The microphotonic device is capacitively coupled with this tissue through a passivation/encapsulation dielectric. A charge will be therefore be induced in the neural tissue, with a quantity dependent on the dielectric thickness and the driving voltage. This charge is electrically coupled to the electrode and will thus cause an artefact.

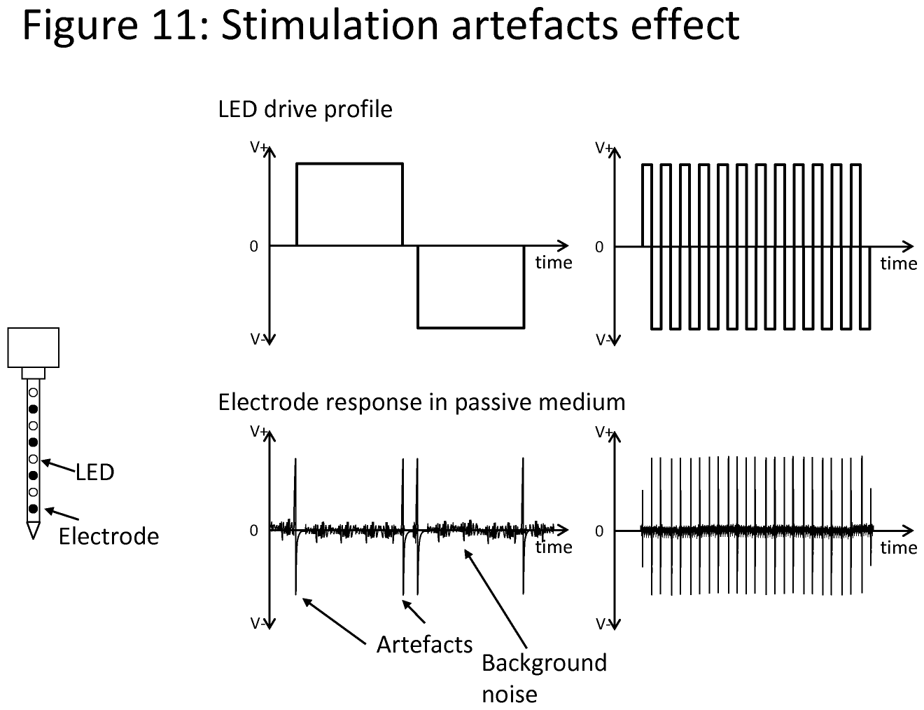

Although the coupling between the LED and the tissue electrolyte is capacitive, it has been demonstrated in the literature that such connections can act be inductive¹⁴. The effect can thus be seen in FIG. 11. Charge induced on the electrolyte surface is picked up by the electrode according to the first derivative of the impulse. As such a purely square wave formation would cause an artefact—which will depend on the dielectric interface. Such artefact could interfere with the electrical recording and thus any therapeutic closed loop ¹⁴ Adela Bardos, Richard N. Zare, Karin Markides, “Inductive behavior of electrolytes in AC conductance measurements”, Chemical Physics Letters 402 (2005) 274-278 control. Whilst it may be possible to filter out such artefacts after signal acquisition, it would be desirable if the artefact from the stimulus can be minimized prior to any filtering.

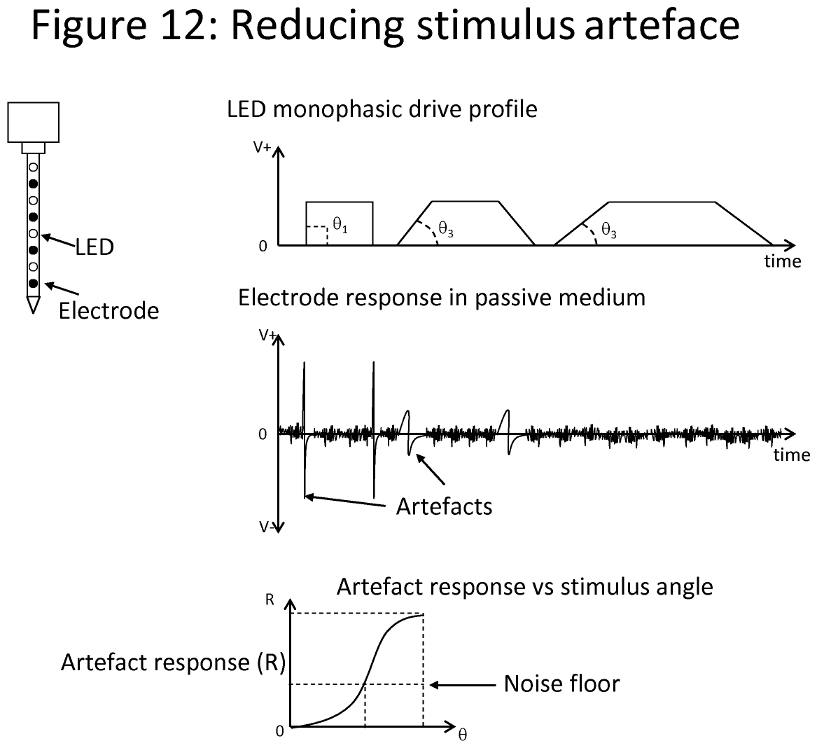

The basic property of inductive AC operation is that that the induced charge varies with the derivative of the electrical field change. As such, a high frequency (step function) change will cause the maximum possible effect, whereas a low frequency (sloped function) will have minimal effect. This is demonstrated in FIG. 12. There will be a slope angle for any device (depending on dielectric properties) whereby the artefact will be effectively below the noise floor for the electrical recording system. Furthermore, if the frequency of the slope function is outside the pass band of the recording amplifier demonstrated in FIG. 10, it will be further attenuated.

As such, any system can have a function of artefact response versus slope angle. Which can be seen in FIG. 13. Thus, for any given noise floor we can define an operating region for slope angles which do not effect the recording signal integrity. This range can be calibrated for different system designs, but the principle will be the same. As such, the maximum frequency of ON-OFF pulsed operation will therefore also be determined by this effect.

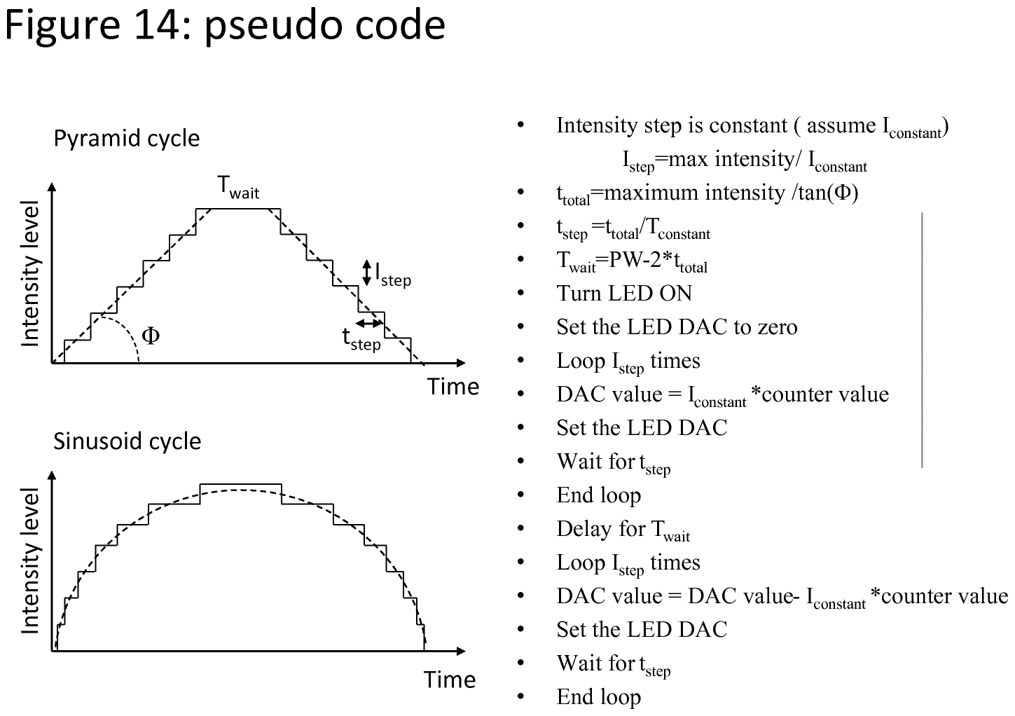

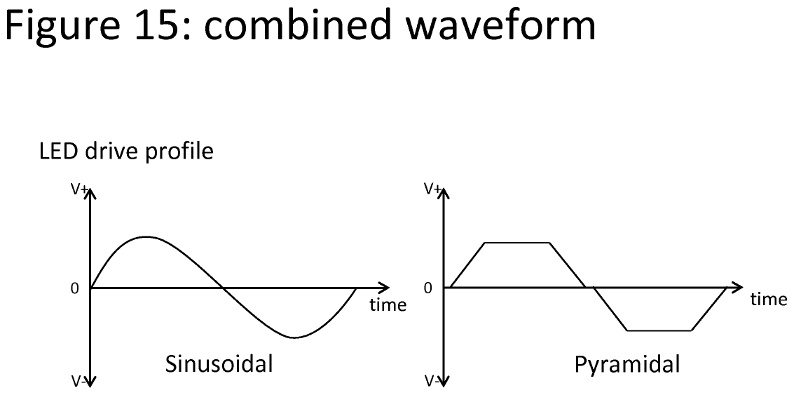

An exemplar implementation and pseudo code can be seen in FIG. 14 for both pyramidal and sinusoidal implementations. Full biphasic balancing can be seen in FIG. 15. Such implementations can be achieved by driving current through the microphotonic device which results in specific voltages across the microphotonic device, and thus between the anode/cathode of that device and the tissue medium. For a typical 8-bit DAC show (included in the stimulation circuit block in FIG. 4), small step functions can be implemented with minimal artefact. There are many ways to implement such digital to analog converters such as Σ□, capacitive and resistive. For a globally implemented DAC, a capacitive approach can provide accuracy. For a locally implemented DAC, Σ□ approaches can provide compactness.

Pseudo code for achieving this functionality in the digital control unit in FIG. 4 is given in FIG. 14. It is clear that there is considerable advantage for having both accurate intensity and timing control. The former allows for stimulus waveforms which minimize artefacts, and the latter (in combination with the former) allows for charge balancing to minimize degradation effects.

The arrangement of processing for such functionality is for a local finite-state-machine in the optrode and an external controller to drive it. An alternative embodiment may be that all digital functionality remains outside the optrode device with analog lines directly controlling passive components. Or another alternative embodiment may be that the digital drive protocol would be fully incorporated either on the optrode digital microelectronics (active probe) or as a bolted-on unit in tandem with the optrode or an optrode array.

The above described control may be achieved by the central control unit presented in FIG. 2, or the digital control unit presented in FIG. 4. In our preferred embodiment, this is implemented in the digital control unit on the optrode (FIG. 4), as it allows for higher speeds, lower latency, reduced energy cost, and does not consume bandwidth on the communication cable.

In practice the effective clock cycle, i.e. when considering it may take more than one clock cycle to implement an action, will determine the cycle time. (c) Describes this effect. Typically the voltage between stimulation and reverse is different. But the minimum cycle ratio is determined by the quantization error from the effective clock. In this case, cycles can be implemented which do not exactly match per cycle, but match over a number of cycles.

The power supply to the optrodes is described in FIG. 2.

A central control unit, typically placed in the chest converts a direct current supply from a battery into an alternating supply which oscillates around tissue ground. Such data/power transmission is common in implantable units as it ensures no net charge dissipation into the tissue in the case of cable rupture. A power conversion system in the optrodes then reproduces a direct current with the tissue ground potential at the centre of the V_{dd} supply and Vₛₛ voltages.

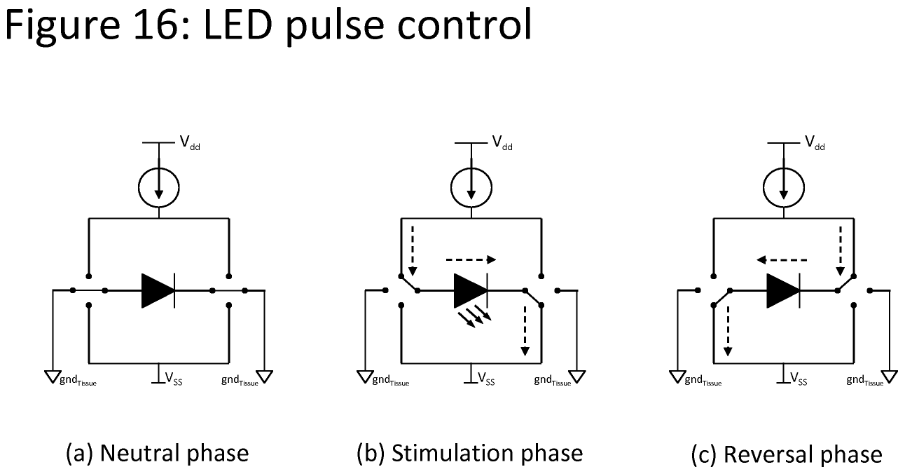

To achieve biphasic operation around the tissue ground point, we utilise a conceptual operation is described in FIG. 16.

A current source determines the level of current flow and thus light output in the reversal phase. Switches then allow for the three phases: stimulation, neutral, and reversal. In the reversal phase (b), current flows in the forward direction through the diode allowing light emission. In the reversal phase (c), current flows in the reverse direction. The current flow, in this case, is very small and related to the leakage properties of the diode. For most emitter configurations, no light is emitted in this phase. In the off state, the voltage across the anode and cathode are clamped to ground. In practice, these switches and the current source are implemented as transistors. The applied voltages across the anode and cathode will then be determined by the configuration of the implemented transistors and the current-voltage profile of the emitter.

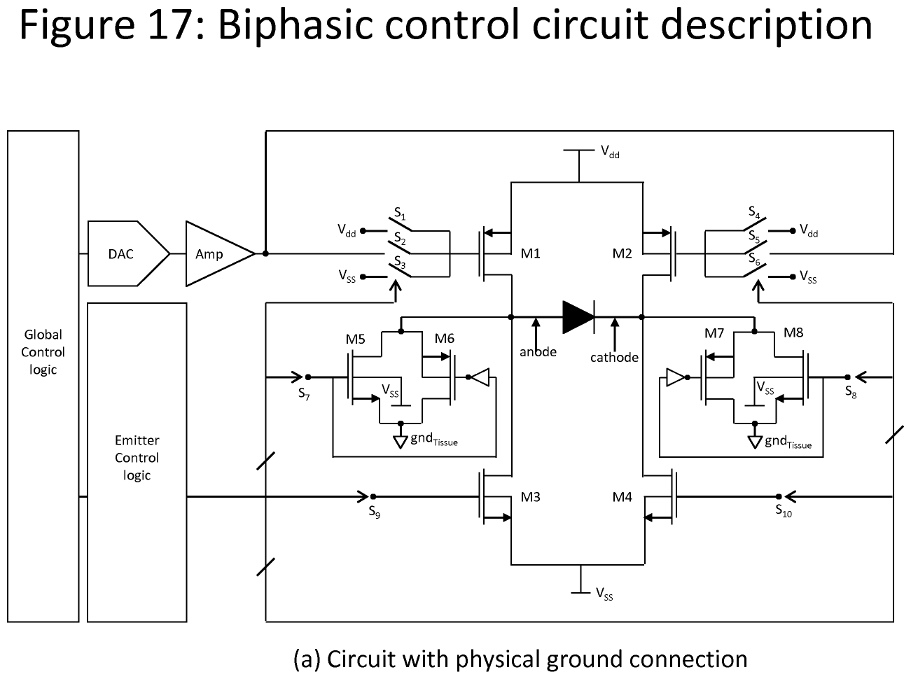

A circuit implementation of this operation is described in FIG. 17. Transistors M1 and M2 act simultaneously as both current sources and switches. The current is defined by a digital to analogue converter (DAC) which is amplified by an inverting voltage amplifier (AMP). AMP is configured with M1 and M2 to provide linear conversion between voltage and current i.e. combined they act as a transconductance amplifier. It can be difficult to achieve the full dynamic range between V_{dd} and Vₛₛ on the output of AMP. Thus Switches S1, S4, and S3, S6 can be used to clamp to respectively to V_{dd} and Vₛₛ, which sets M1 and M2 respectively fully OFF and fully ON.

Transistors M3 and M4 act as switches to mediate current flow in the stimulation and reversal phases. During operation both transistors will generally be in the triode operational region as much of the V_{dd}-Vₛₛ voltage will drop across the diode constraining the source-drain voltage of the transistors. As such, they act more precisely like voltage controlled variable resistors than ideal current sources.

M5-M8 allow clamping the anode and cathode to tissue ground. These can be included if the resting potential of the anode and cathode without such structures deviates from tissue ground. This is typically the case if M1-M4 are not the same size. The reverse current through M2 will differ from the stimulation current through M1. Thus, the W/L ratios of these transistors are different. Furthermore, the midpoint of V_{dd} and Vₛₛ as defined in FIG. 2 may not exactly match gnd_{Tissue}.

If the deviation from tissue ground is small, M6 and M7 can be negated. Additionally, if a triple well or SOI CMOS process can be used with an isolated n-well, then the body of M5 and M8 should be connected to tissue ground to prevent a body effect. Switches S1 to S10 represent digital switches which can be implemented as transistors. For some ‘AMP’ implementations, it may be difficult to achieve the maximum values of V_{dd} and Vₛₛ to correspond with the DAC values 0 and 255. Thus, S₁ and S₄, can be used to clamp to V_{dd}, and S₃ and S₆ to clamp to Vₛₛ.

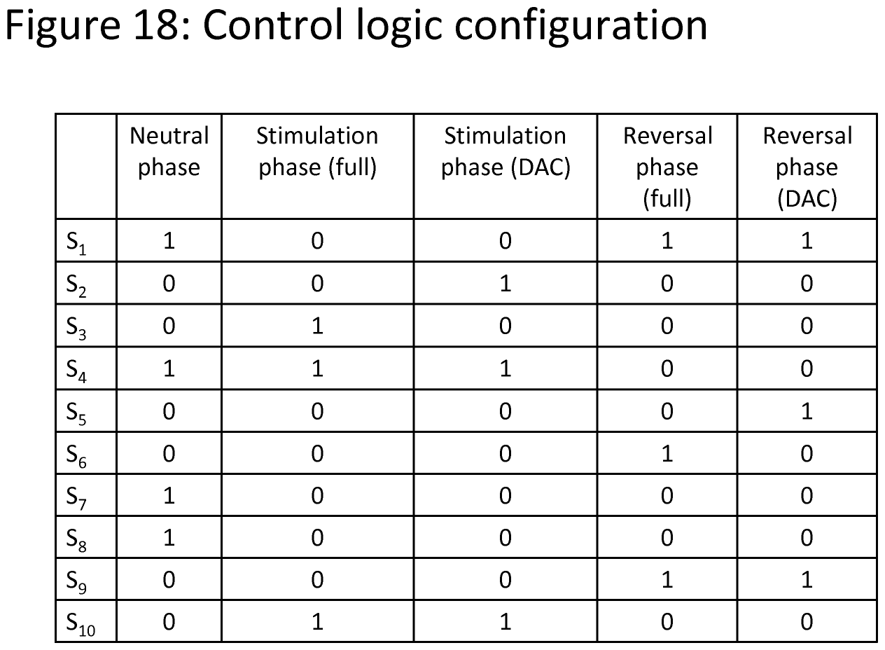

The stimulation and reverse phases can be achievable by switching the related transistors on or off. For example, the stimulation phase requires M2 and M3 (and M5-M8) transistors to be off and M1 and M4 transistors to be ON. The cycle time can be achieved through rapid ON-OFF switching with the integral ON time providing pulse width modulation. Furthermore, current control will be done using DAC and through TCA. In reverse mode, M1 and M4 are off, and M2 and M3 are controlled by control logic for PWM and DAC for current control. The full table of operation is provided in FIG. 18.

Light emission in the configuration presented in FIG. 16 may be controlled by both current and time (Pulse Width Modulation—PWM). Alternate simplified configurations could modulate pulse widths (PWM) for a fixed current, or modulate current for fixed pulse widths. The operational setting of the DAC and the PWM is determined by the global control system.

To minimize degradation effects, the [voltage×time] of the anode in the stimulation phase must equal the −[voltage×time] in the reversal phase. The same must be true of the cathode. To achieve such balancing operation, two different strategies may be used:
(1) Changing the DAC value in the reverse state to match the voltage. This allows for time matching of the forward and reverse pulse. This is only feasible in optical elements with limited rectification.

(2) Changing the time period of the reversal phase to match the integral function of the voltage in the stimulation phase with time. Although the stimulation/reversal cycle can be operated at sub-millisecond, the practical time will be a function of the required matching accuracy and the minimum switch time.

The local state switching settings are defined in a table in FIG. 18. The specific PWM timings can be determined as appropriate in the global control logic, which is typically implemented as a finite state machine. The limitation on the PWM accuracy is determined by the internal clock which is typically in the MHz domain. As the LED illumination pulse widths are typically between 0.1-10 ms, accuracies of >99% are achievable in balancing [voltage×time] for the stimulation and reversal phases.

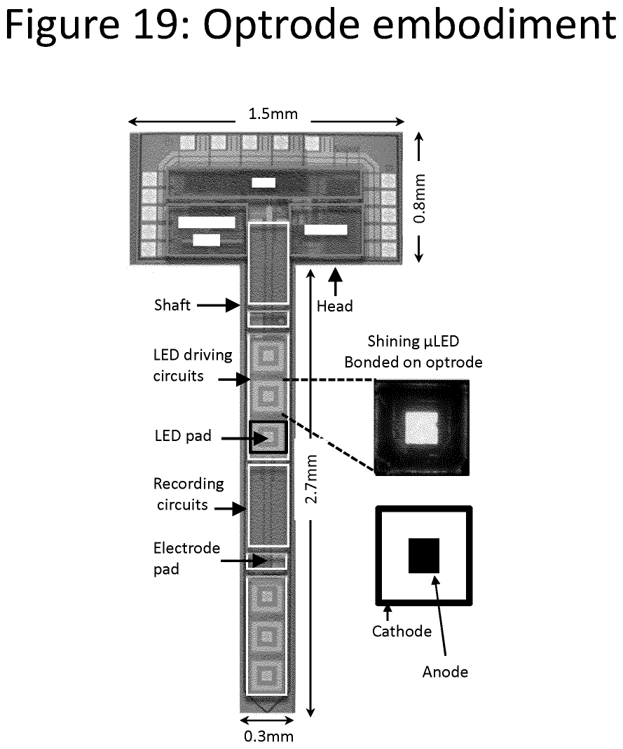

FIG. 19 describes a fully fabricated optrode with the capabilities described above. In this case, the optrode is manufactured from a complementary metal oxide semiconductor (CMOS) substrate. Gallium nitride micro-LEDs with a centre surround format have been bonded on-top. The inset image describes the illumination thereof.

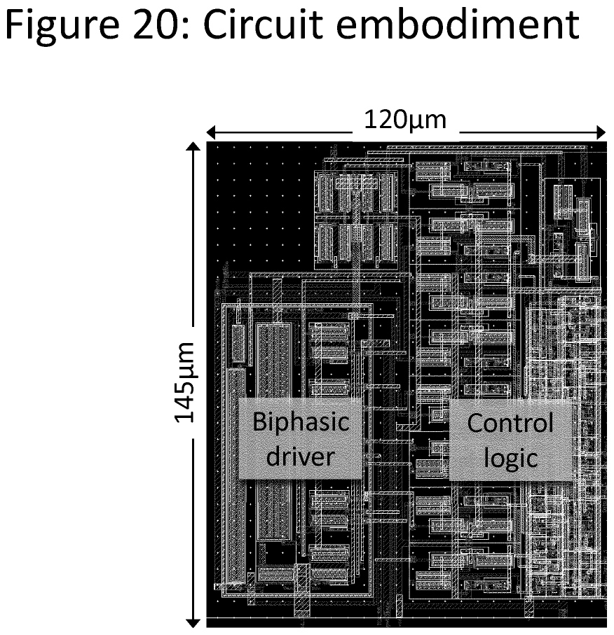

FIG. 20 describes the computer aided design layout of the emitter control circuit in 0.35 μm CMOS technology and forms part of the optrode described in FIG. 12. Alternate technology nodes can compact this circuit further.

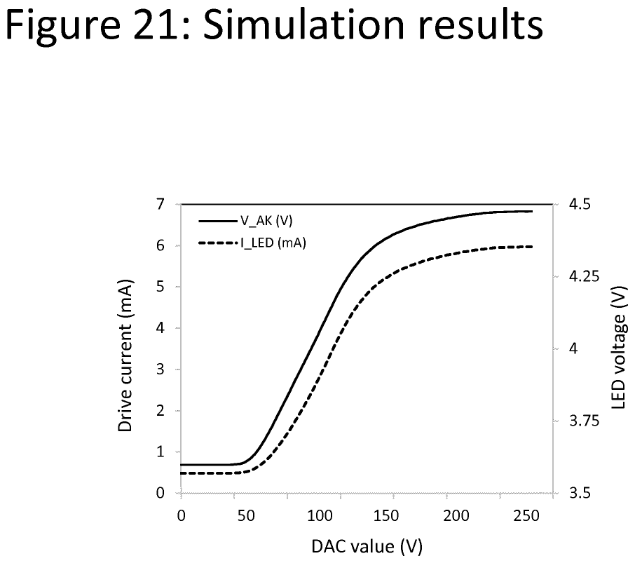

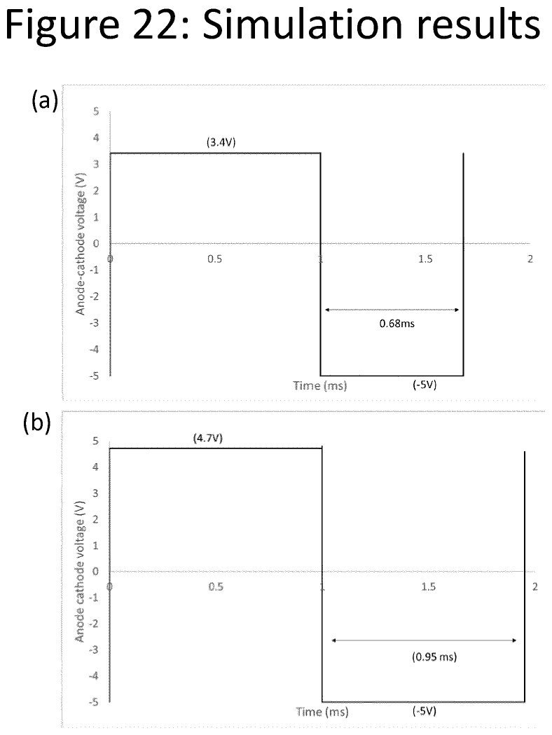

FIG. 21 displays the simulation results achieved for the driver circuit which provides a large output voltage (up to 4.5V) and current (up to 6 mA) across the LED anode-cathode. FIG. 22 shows simulation results of light emitting diode biphasic operation. (a) shows the results for driving 1 mA through our circuit balancing 3.4×10⁻³ Vs and (b) for driving 3.4 mA, and balancing 4.7×10⁻³vs.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

All references referred to above and below are hereby incorporated by reference.

### References

1. Cogan, S. F., Neural stimulation and recording electrodes, in Annual Review of Biomedical Engineering. 2008. p. 275-309.
2. Nagel, G., et al., Channelrhodopsin-2, a directly light-gated cation-selective membrane channel. Proceedings of the National Academy of Sciences of the United States of America, 2003. 100(24): p. 13940-13945.
3. Grossman, N., et al., Modeling Study of the Light Stimulation of a Neuron Cell With Channelrhodopsin-2 Mutants. Ieee Transactions on Biomedical Engineering, 2011. 58(6): p. 1742-1751.
4. Beiert, T., T. Bruegmann, and P. Sasse, Optogenetic activation of G(q) signalling modulates pacemaker activity of cardiomyocytes. Cardiovascular Research, 2014. 102(3): p. 507-516.
5. Koyanagi, M. and A. Terakita, Diversity of animal opsin-based pigments and their optogenetic potential. Biochimica Et Biophysica Acta-Bioenergetics, 2014. 1837(5): p. 710-716.
6. Tian, L., et al., Imaging neural activity in worms, flies and mice with improved GCaMP calcium indicators. Nature Methods, 2009. 6(12): p. 875-U113.
Knopfel, T., et al., Toward the Second Generation of Optogenetic Tools. Journal of Neuroscience, 2010. 30(45): p. 14998-15004.
8. Son, Y., et al., In vivo optical modulation of neural signals using monolithically integrated two-dimensional neural probe arrays. Scientific Reports, 2015. 5: p. 11.
9. McAlinden, N., et al., Optogenetic activation of neocortical neurons in vivo with a sapphire-based micro-scale LED probe. Frontiers in Neural Circuits, 2015. 9.
10. McAlinden, N., et al., Thermal and optical characterization of micro-LED probes for in vivo optogenetic neural stimulation. Optics Letters, 2013. 38(6): p. 992-994.
11. McGovern, B., et al., A New Individually Addressable Micro-LED Array for Photogenetic Neural Stimulation. Ieee Transactions on Biomedical Circuits and Systems, 2010. 4(6): p. 469-476.
12. Eisenbrand, F., A. Karrenbauer, and C. H. Xu, Algorithms for longer OLED lifetime, in Experimental Algorithms, Proceedings, C. Demetrescu, Editor. 2007, Springer-Verlag Berlin: Berlin. p. 338-351.
13. Lin, C. L., et al., Lifetime Amelioration for an AMOLED Pixel Circuit by Using a Novel AC Driving Scheme. Ieee Transactions on Electron Devices, 2011. 58(8): p. 2652-2659.
14. Fan, Z. Y., J. Y. Lin, and H. X. Jiang, III-nitride micro-emitter arrays: development and applications. Journal of Physics D-Applied Physics, 2008. 41(9).
15. Maaskant, P. P., et al., High-Speed Substrate-Emitting Micro-Light-Emitting Diodes for Applications Requiring High Radiance. Applied Physics Express, 2013. 6(2).
16. Degenaar, P., et al., Optobionic vision-a new genetically enhanced light on retinal prosthesis. Journal of Neural Engineering, 2009. 6(3).
17. Zhang, H. X., et al., Individually-addressable flip-chip AlInGaN micropixelated light emitting diode arrays with high continuous and nanosecond output power. Optics Express, 2008. 16(13): p. 9918-9926.
18. Zhao, H. B., et al., An Implantable Optrode with Self-diagnostic Function in 0.35 mu m CMOS for Optical Neural Stimulation, in 2014 Ieee Biomedical Circuits and Systems Conference. 2014. p. 244-247.
19. Cao, H., et al., An Integrated mu LED Optrode for Optogenetic Stimulation and Electrical Recording. Ieee Transactions on Biomedical Engineering, 2013. 60(1): p. 225-229.
20. Clements, I. P., et al., Miniaturized LED sources for in vivo optogenetic experimentation, in Optogenetics: Optical Methods for Cellular Control, S. K. Mohanty and N. V. Thakor, Editors. 2013.
21. Adela Bardos, Richard N. Zare, Karin Markides, “Inductive behavior of electrolytes in AC conductance measurements”, Chemical Physics Letters 402 (2005) 274-278

1. An optical stimulation arrangement including:
a light-emitting device, implantable in an environment with an associated ground voltage, the light emitting device including:
a light emitting element;
an anode; and
a cathode; and
a controller for driving the light-emitting device in a biphasic manner.

2. (canceled)

3. An optical stimulation arrangement according to claim 1, wherein the light-emitting element is a semiconductor light-emitting element comprising a diode, a laser, or a light emitting diode with laser like properties.

4. An optical stimulation arrangement according to claim 1, wherein the light-emitting device has a plurality of contacts.

5. An optical stimulation arrangement according to claim 4, wherein the light-emitting device has 3 or 4 or more contacts, wherein the light-emitting device is a light-emitting transistor.

6. (canceled)

7. An optical stimulation arrangement according to claim 1, wherein the light-emitting element comprises of organic, inorganic or quantum dot substrates.

8. An optical stimulation arrangement according to claim 1, wherein the controller is configured to drive the light-emitting device with a stimulation phase and a reversal phase, each having an associated voltage-time profile.

9. An optical stimulation arrangement according to claim 8, wherein the voltage-time profile associated with the reversal phase is selected to balance out the voltage-time profile associated with the stimulation phase.

10. An optical stimulation arrangement according to claim 8, wherein the light-emitting element is in an ON state during the stimulation phase.

11. An optical stimulation arrangement according to claim 8, wherein the integral of the voltage-time profile of the reversal phase is equal or substantially equal to the negative of the integral of the voltage-time profile associated with the stimulation phase.

12. An optical stimulation arrangement according to claim 8, wherein the stimulation and reversal phases are interleaved at high frequency to minimize the stimulation/reversal cycle time without causing artefact.

13. An optical stimulation arrangement according to claim 12, comprising an algorithm implemented on a digital control to compensate for neuron adaptation due to the total stimulus time for interleaved cycles greater than a single cycle.

14. An optical stimulation arrangement according to claim 12, wherein the integral function of the stimulation sub-cycles matches the required integral stimulation time for optical stimulus of the target biological/molecular structures.

15. An optical stimulation arrangement according to claim 8, wherein the integral stimulus time or stimulus reversal cycle time is 1 μs- 100 ms

16. (canceled)

17. An optical stimulation arrangement according to claim 8, wherein the stimulus-reversal cycle time is implemented as a series of pulses matching the effective clock cycle, whereby individual sub-cycles are not fully matched, but the overall cycle time is.

18. An optical stimulation arrangement according to claim 8, wherein the controller includes control circuitry configured to switch between the stimulation phase and the reversal phase, wherein the control circuitry includes at least one switch and a current source, wherein one or more of the at least one switch and the current source is/are implemented using one or more transistors.

19. An optical stimulation arrangement according to claim 8, wherein the controller is further configured to drive the light-emitting device in a neutral phase, in which the voltage across the anode and the cathode is the same as, or substantially the same as, the ground voltage associated with the surrounding environment.

20. An optical stimulation arrangement according to claim 19, wherein the controller includes control circuitry configured to switch between the stimulation phase, the neutral phase and the reversal phase, wherein the control circuitry includes at least one switch and a current source, wherein one or more of the at least one switch and the current source is/are implemented using one or more transistors.

21-23. (canceled)

24. An optical stimulation arrangement according to claim 18, wherein the control circuitry includes means for measuring the ground voltage associated with the surrounding environment, wherein the means for measuring is implemented using one or more transistors.

25. An optical stimulation arrangement according to claim 20, wherein the control circuitry includes means for measuring the ground voltage associated with the surrounding environment, wherein the means for measuring is implemented using one or more transistors.

26. An optical stimulation arrangement according to claim 1, wherein the environment is body tissue, and the associated ground voltage is a tissue ground voltage.

27. An optical stimulation arrangement according to claim 26, wherein the body tissue is neural tissue, such as brain tissue or retinal tissue.

28. An optical stimulation arrangement according to claim 26, wherein the light-emitting device includes one or more optrodes or other implantable probes, further comprising local control electronics for the light-emitting device, the light-emitting device and local control electronics being implemented onto the same optrode or the same other implantable probe.

29. (canceled)

30. An optical stimulation arrangement according to claim 1, wherein the controller is located in a central control unit, which is connected to the light-emitting device.

31. An optical stimulation arrangement according to claim 30, wherein the central control unit is connected to the light-emitting device via:
connective leads; or
a wireless connection.

32. An optical stimulation arrangement according to claim 30, wherein the central control unit either includes a power supply, or is configured to receive a power supply.

33. An optical stimulation arrangement according to claim 30, wherein the central control unit is implantable into a user.

34. An optical stimulation arrangement according to claim 1, wherein the controller includes means for wirelessly communicating with external electronic devices, wherein the wireless communication is either Bluetooth or Wi-Fi based and/or falls within accepted ISM or Medradio bands.

35. (canceled)

36. An optical stimulation arrangement according to claim 1, wherein the derivate change in voltage across the light emitting device is modulated in steps such that no artefact is generated in the tissue.

37. An optical stimulation arrangement according to claim 36, wherein a positive and/or negative microphotonic drive profile for the light emitting device is implemented as:
a pyramid function;
a trapezoid function; or
a sinusoidal function,
wherein the positive and/or negative phases of the drive profile have equal integral functions.

38-39. (canceled)

40. An optical stimulation arrangement according to claim 1, wherein a slope profile for achieving minimal artefact is stored as a look up table in a control system, the control system configured to store a series of commands to control operation of the light-emitting device, the control system comprising:
a microelectronic control unit on the light-emitting device; or
a control unit that is external to the light-emitting device.

41-42. (canceled)

43. An optical stimulation arrangement according to claim 1, wherein the light-emitting device is stimulating optogenetic cells in the range 470 nm-650 nm.

44. An optical stimulation arrangement according to claim 1, wherein the light-emitting device is stimulating optogenetic or non-optogenetic fluorophores in the range 470 nm-800 nm.

45. An optical stimulation arrangement according to claim 1, wherein the light-emitting device is emitting in the near infra-red for diagnostic purposes.

46. An optical stimulation arrangement according to claim 1, wherein the light-emitting device is emitting short blue pulses for autofluorescence diagnosis.
